(19) United States
(12) Patent Application Publication
(10) Pub. No.: US 2025/0128994 A1
(43) Pub. Date: Apr. 24, 2025

(54) METHOD OF USING/APPLYING A KERATIN HYDROLYSIS PEPTIDE SOLUTION TO PROMOTE THE GROWTH AND DEVELOPMENT OF CORN CROPS

(71) Applicant: CH Biotech R&D Co., Ltd., Nantou City (TW)

(72) Inventors: Kai XIA, Nantou (TW); Ya-Wen HUANG, Taichung City (TW); Gui-Jun LI, Taichung (TW); Nai-Hua YE, Changhua (TW)

(73) Assignee: CH Biotech R&D Co., Ltd., Nantou City (TW)

(21) Appl. No.: 18/412,611

(22) Filed: Jan. 15, 2024

(30) Foreign Application Priority Data
Oct. 20, 2023 (TW) 112140294

(51) Int. Cl. C05F 1/00; C07K 14/47

(52) U.S. Cl. CPC C05F 1/005; C07K 14/4741

(57) ABSTRACT

Present invention teaches the method of using a keratin hydrolysis peptide (“KHP”) solution to promote the growth and development of corn crops, by selectively choosing specific weights of feathers and water, and treating the mixture to a high-temperature high-pressure hydrolysis process, the resulting solution is confirmed to contain at least 253 peptides and, at seedling stage and/or early growth stage, applied to the soil around or sprayed to the young plants. Optionally, the KHP solution can be diluted by water, as disclosed in the specification, before administering as taught herein.

### PRIORITY CLAIM TO FOREIGN APPLICATION

Applicant hereby makes priority claim to a Taiwan application, number 112140294, having the Taiwan filing date of Oct. 20, 2023.

### BACKGROUND OF THE INVENTION

Present invention disclosed and claimed the method and application of a keratin hydrolysis peptide (“KHP”) solution to corn plants, in order to promote better growth and yield of corn crops.

The KHP solution is made by a hydrolysis process upon a mixture of feathers and water, at different weight ratio, via a high-temperature and high-pressure process, resulting in a solution that has many beneficial applications in the fields of horticulture, agriculture and potentially other farming businesses.

The KHP solution made pursuant to the disclosure herein is applied to corn plants/crops at sowing, seedling and early growth stages, and helps to increase root system and shoot weight of young corn plants, improving photosynthetic capacity and nutrient-intake of the corn plant, resulting in better growth and development.

Hydrolyzed keratin has long been used to strengthen hairs, reduce breakage, and minimize damages. Some form of chemical process can be used to break down the large protein molecule of the hydrolyzed keratin so that it can be better absorbed and utilized to benefit the hair and other tissues. However, due to potential environment concerns, the breakdown by chemical process is not well received in the agricultural community.

In the ever increasingly urgent context of climate changes that lead to severe agricultural losses, and in light of the projected world population reaching 9 billion by the year 2050, it is imperative that the agricultural production has to increase dramatically to feed the population of the world. Among the key agricultural crops is the corn crops, which are among the most important food sources for people worldwide.

People have been using fertilizers to boost the corn production for many years. It is well known that nitrogen-based fertilizers are directly tied to the growth the production of corn crops. Some studies also showed that, given the use of the nitrogen-based fertilizers, if there is an addition boost to the corn plant's growth and development, the fertilizers' intake and utilization efficacy would increase substantially, resulting in cost-effective production of the corn crops.

Particularly, using a KHP solution to the corn crop, based upon the invention stated herein, greatly increase the growth and development of corn plants that are already aided by the nitrogen-based fertilizers.

By the inventors' tests conducted, it was proved that the use of the KHP solution enhanced the growth of leaf surface and the content of chlorophyll, thus added to the capacity of the plants' photosynthesis function, and ultimately resulting in the better health of the corn plants and production yield.

### SUMMARY OF THE INVENTION

It is known that feather contains 85-91% keratin, 13-15% organic nitrogen, 1.6-2% organic sulfur, as well as other materials. The high keratin content has drawn many prior researches that work to break down, by enzyme, chemical agents, or fermentation process, into peptides, amino acids and other smaller molecules that can be used for animal feeds, plant fertilizers, and cultivation bases.

Around 2019, Nurdiawati, et al, came up with a hydrolysis process, by the mixture of α-amylase and protease to hydrolyze feather waste, resulting in a mixture of amino acids, fatty acids, and sugars. Nurdiawati experimented and adopted certain specific high-temperature and high-pressure setting in the hydrolysis process and discovered that the resulting solution, when mixed with some potassium and other minerals, can boost the growth of Pogostemon cablin and Vigna radiata, as reported in International Journal of Recycling or Organic Waste in Agriculture (8:221-232, 2019).

The inventors of present application, under the aegis of CH Biotech, developed a method of creating a keratin solution and method of applying the solution to the corn plants, and proved by experiments that such a method in fact helped with the increase of corn plant's health and production.

In an embodiment method of the KHP solution to promote the growth and development of corn crops, consisting the steps of:
a. Preparing the KHP solution by mixing 66 kg of feathers whose content is 50% water and 44 kg of water in a sealed container;
b. hydrolyzing the mixture in the container with a temperature and pressure setting of 195° C. and 16 kg/cm² for a duration of 40 minutes;
c. using a mass spectrometer to confirm the combination of peptides in the solution to contain at least 253 peptides as listed in the specification where their molecular masses are between 500 and 4,000 Daltons, and the concentration is in the range of 2.0×10⁵˜4.5×10⁵ ppm;
d. applying the solution to the soil around corn plants at seedling stage.

To confirm the 253 peptides, the inventors used a Dionex UltiMate 3000 UPLC to separate the peptides; an analysis is done via Thermo Orbitrap Fushion Lumos Tribrid Orbitrap mass spectrometry to identify the peptides, which are then subsequently confirmed by looking up the BIOPEP-UWM database.

The method of using a keratin hydrolysis peptide (KHP) solution stated above where the solution is sprayed to the corn plants at early growth stage.

The method of using a keratin hydrolysis peptide (KHP) solution stated above where the solution is diluted with water by volume at the ratio of 1:50-1000 and sprayed to the corn plants at the early growth stage.

The method of using a keratin hydrolysis peptide (KHP) solution stated above where the solution is diluted with water by volume at the ratio of 1:100-500 for applying to the corn plants.

### BRIEF DESCRIPTION OF DRAWINGS

The accompanying drawings, figures and tables, which are incorporated in and constitute a part of this specification, illustrate and exemplify the preferred embodiments of the invention. Together with the description, serve to explain the principles of the invention.

Table I shows the at least 253 peptides and its annotated sequences for the solution generated in accordance with the disclosure of this application.

FIG.1 shows the dry weights of the roots for the corn plants in the three groups: CK, KHP-1 100× and KHP-1 50×.

FIGS. 2A and 2B show the leaf surface area comparisons among the three groups.

FIG. 2C shows the chlorophyll count by SPAD readings of the three groups.

FIG. 3 shows the chlorophyll count by SPAD readings of the CK group vs the KHP-1 group at the young shoot stage.

FIGS. 4A, 4B, 4C and 4D show the above-ground stem's height, width, net weight and dry weight comparisons among the three groups

FIGS. 5A, 5B, 5C and 5D compare one KHP-1 100× group to the check group (water only), to show the differences in the corn plants between growth status (image), above-ground dry weight, overall height, and stem width.

### DETAILED DESCRIPTION OF THE INVENTION

The keratin hydrolysis peptide (“KHP”) solution of present invention has two embodiments based upon the weight ratio between feathers and water. One embodiment uses 66 kg of feathers mixed with 44 kg of water; the other embodiment uses 50 kg of feathers mixed with 40 kg of water. The feathers have a water content of 50%.

The mixture then is treated by a high-temperature and high-pressure process as further described below.

In a first embodiment method of the KHP solution to promote the growth and development of corn crops, consisting the steps of:
a. Preparing the KHP solution by mixing 66 kg of feathers whose content is 50% water and 44 kg of water in a sealed container;
b. hydrolyzing the mixture in the container with a temperature and pressure setting of 195° C. and 16 kg/cm² for a duration of 40 minutes;
c. using a mass spectrometer to confirm the combination of peptides in the solution to contain at least 253 peptides as listed in the specification where their molecular masses are between 500 and 4,000 Daltons, and the concentration is in the range of 2.0×10⁵˜4.5×10⁵ ppm;
d. applying the solution to the soil around corn plants at seedling stage.

To confirm the 253 peptides, the inventors used a Dionex UltiMate 3000 UPLC to separate the peptides; an analysis is done via Thermo Orbitrap Fushion Lumos Tribrid Orbitrap mass spectrometry to identify the peptides, which are then subsequently confirmed by looking up the BIOPEP-UWM database.

The method of using a keratin hydrolysis peptide (KHP) solution stated above where the solution is sprayed to the corn plants at early growth stage.

The method of using a keratin hydrolysis peptide (KHP) solution stated above where the solution is diluted with water by volume at the ratio of 1:50-1000 for applying to the soil planted with corn seedlings or spraying to the corn plants at the early growth stage.

The method of using a keratin hydrolysis peptide (KHP) solution of claim 1 where the solution is diluted with water by volume at the ratio of 1:100-500 for applying to the soil planted with corn seedlings or spraying to the corn plants at the early growth stage.

The two embodiments' hydrolysis parameters are shown in the table herein; though the claims and tests are focused on first embodiment; the KHP solution made pursuant to the parameters in the first embodiment is referred to as KHP-1.

| | | | Water | | | | | |
|---|---|---|---|---|---|---|---|---|
| | | | Content | | | | | |
| | Feather | Water | in | | | | | |
| | Weight | Weight | Feathers | Pressure | Temp | Time | Mass | Concentr. |
| | (kg) | (kg) | % | (kg/cm²) | (° C.) | (min) | (Dalton) | (ppm) |
| KHP-1 | 66 | 44 | 50% | 16 | 195 | 40 | 593.3~3828.0 | 200000 |
| | 50 | 40 | 50% | 12 | 185 | 80 | 593.3~3508.9 | 301500 |

To confirm the effectiveness of the KHP solution application to the corn crops, three groups of corn crops are defined for conducting tests: a check group (CK) is given water and normal nutrients (including normal nitrogen-based fertilizers) and growth environment; the other groups are denoted by the solution used. The group of KHP-1 100× denotes applying with the KHP-1 solution diluted by water volume of 1:100; the group of KHP-1 50× denotes applying with the KHP-1 solution diluted by water volume of 1:50.

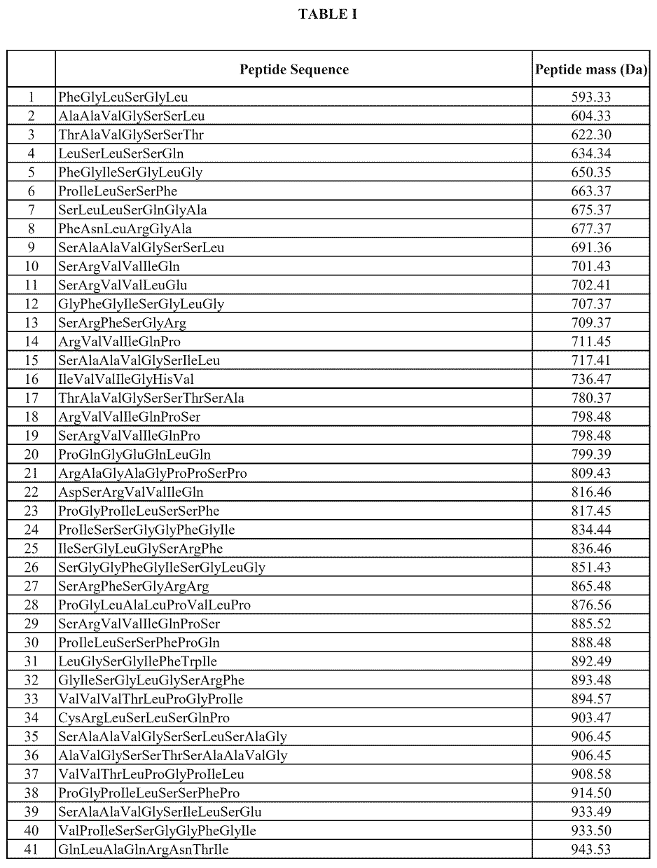

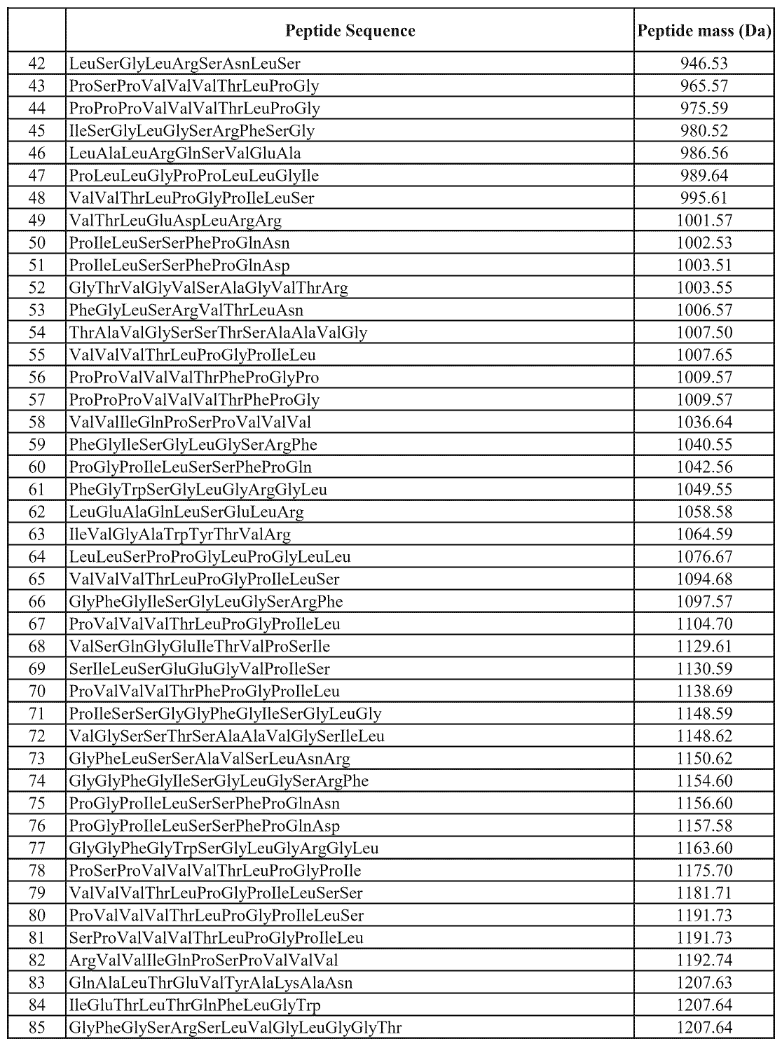

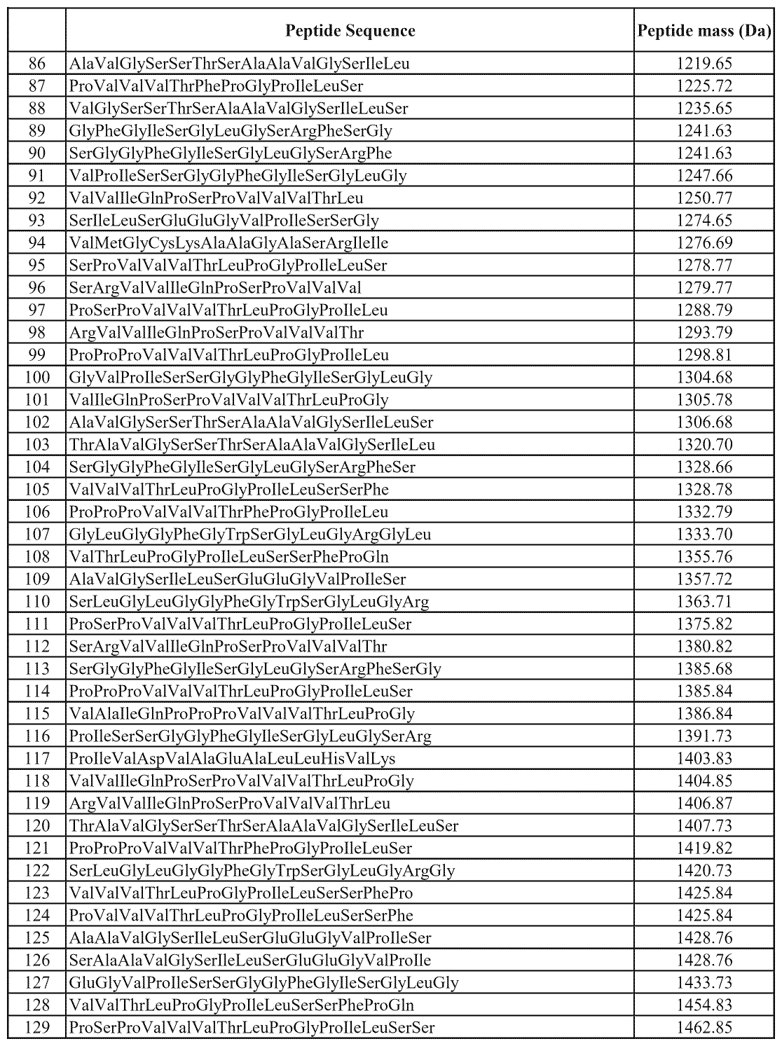

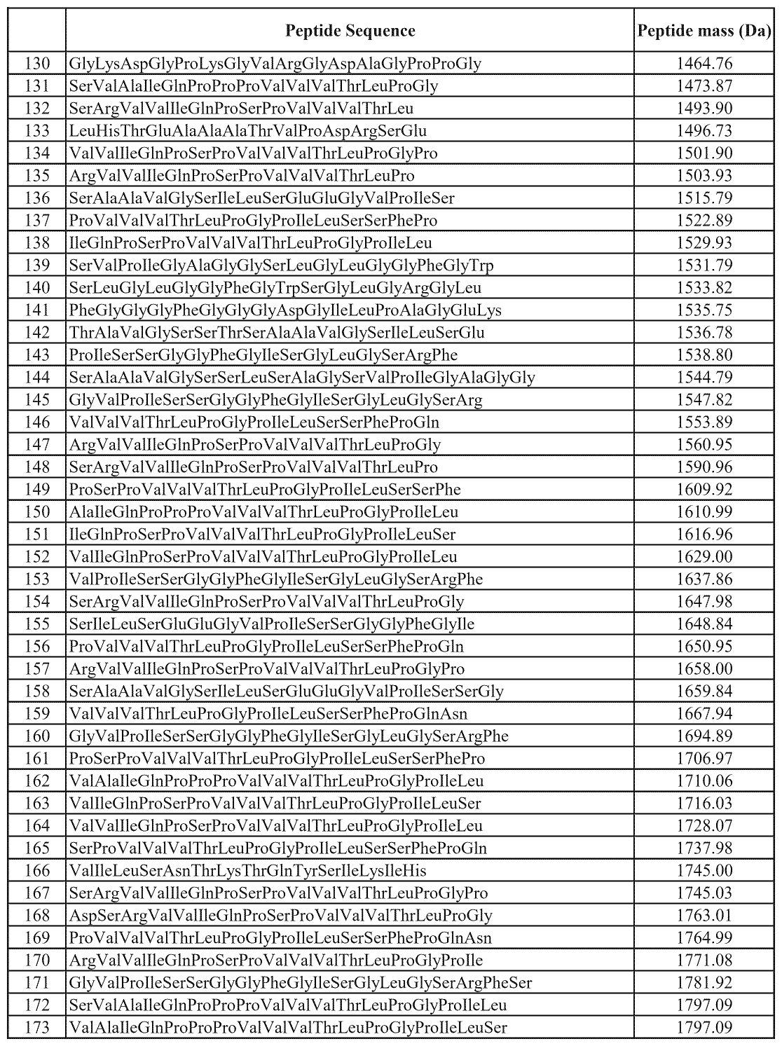

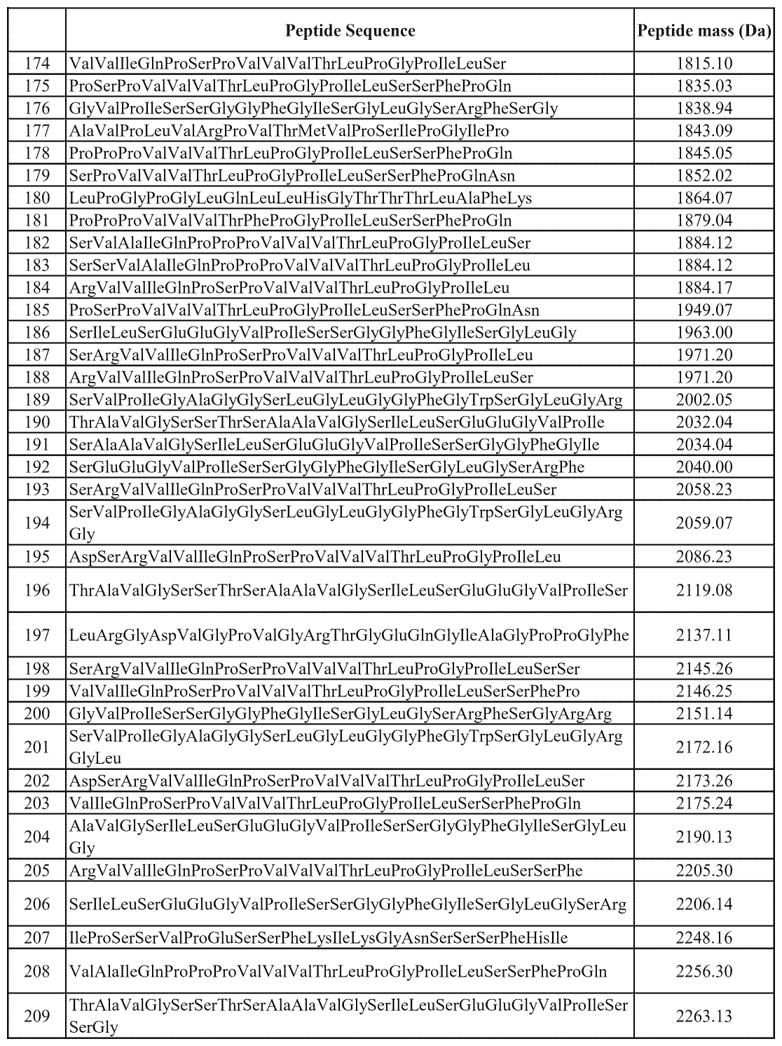

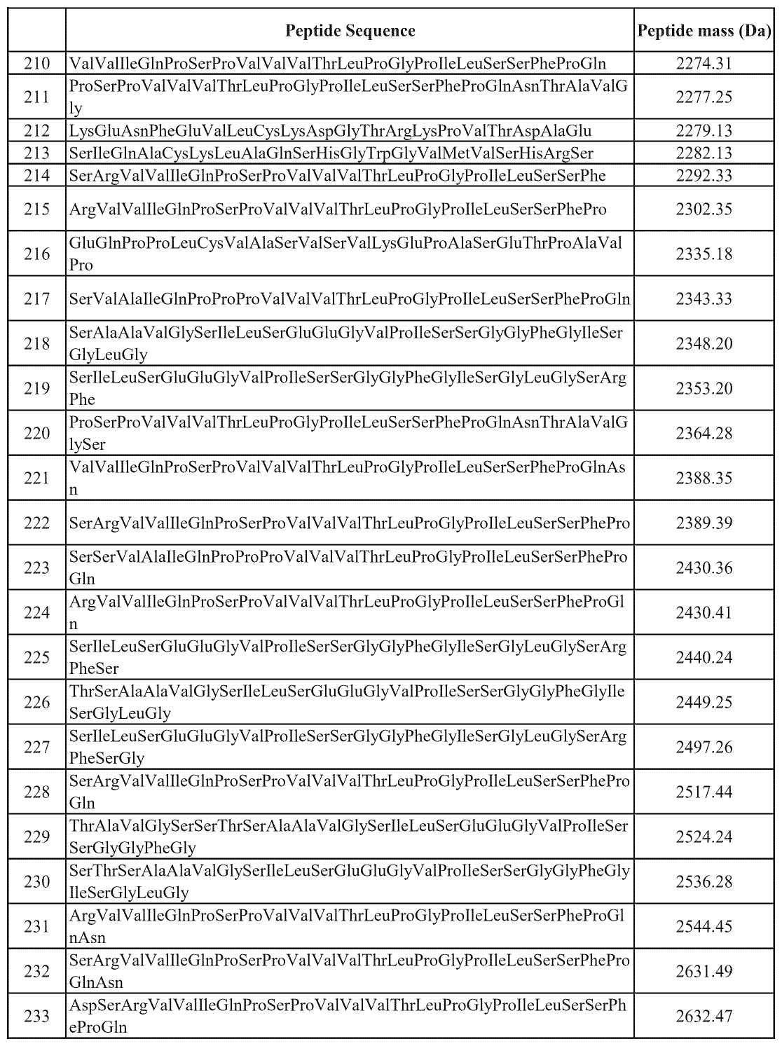

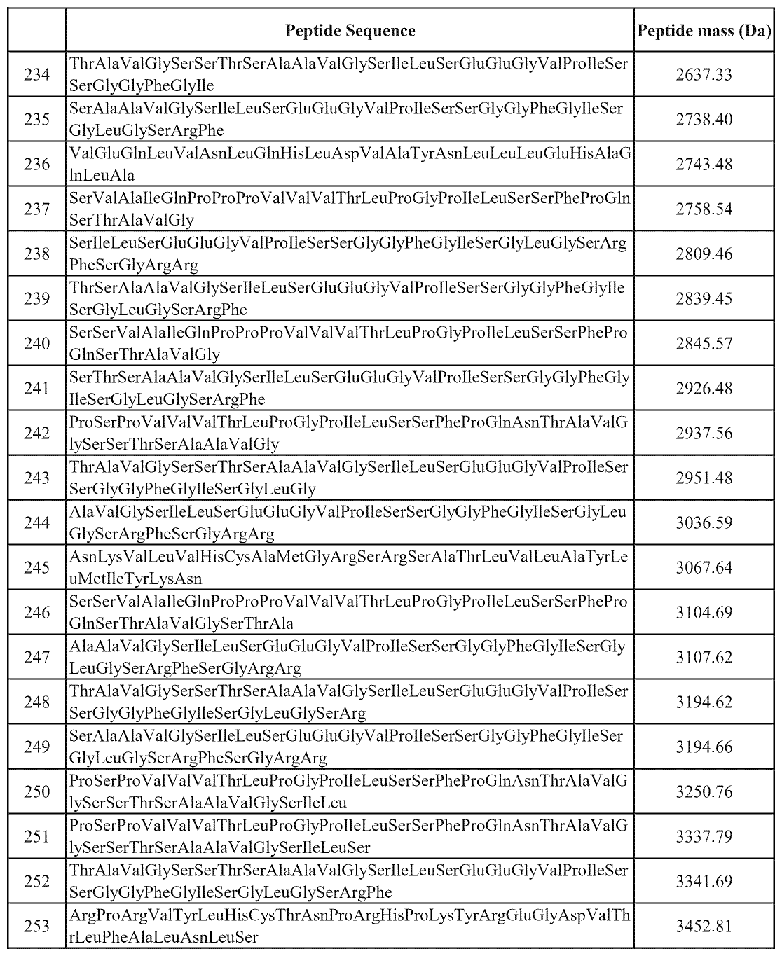

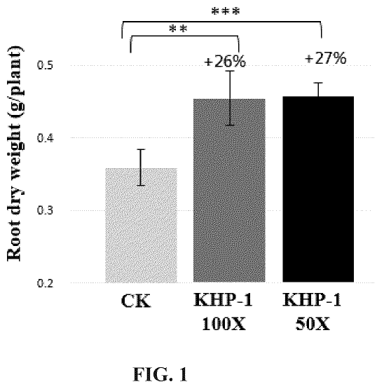

In the tests done with green-house like environment, the corn seedlings are planted into soil infused with the KHP-1 solutions, both 100× and 50× versions. After 14 days, the roots from the three groups are taken out, cleaned and measured the dry weight. The results are noted in FIG.1. The root dry weights for the two KHP-1 groups (100× and 50×) are 26% and 27% heavier than the CK group.

The inventors further test the chlorophyll counts of the seedlings planted into soil infused with the KHP-1 solutions, both 100× and 50× versions.

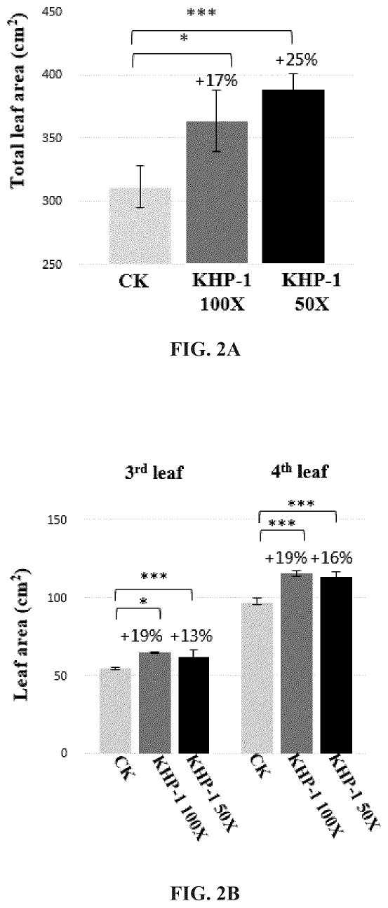

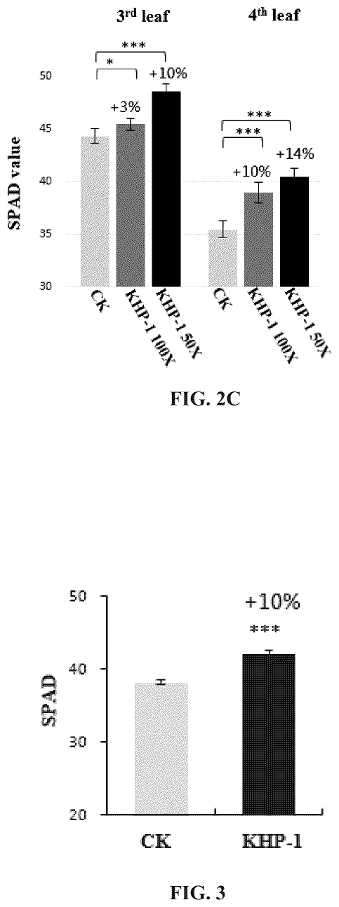

After 14 days, the leaf areas (specifically picking third and fourth leaves) are measured by a WinFOLIA Pro LA2400 system. The comparisons of the leaf surface are noted in FIGS. 2A and 2B. The SPAD chlorophyll counts are noted in FIG. 2C.

At young shoot stage, 12 days after the corn plants are sprayed with the KHP-1 solution, the fifth leaf is specifically taken to measure the chlorophyll counts by the SPAD 502 plus machine. The result (CK vs KHP-1) is shown in FIG. 3.

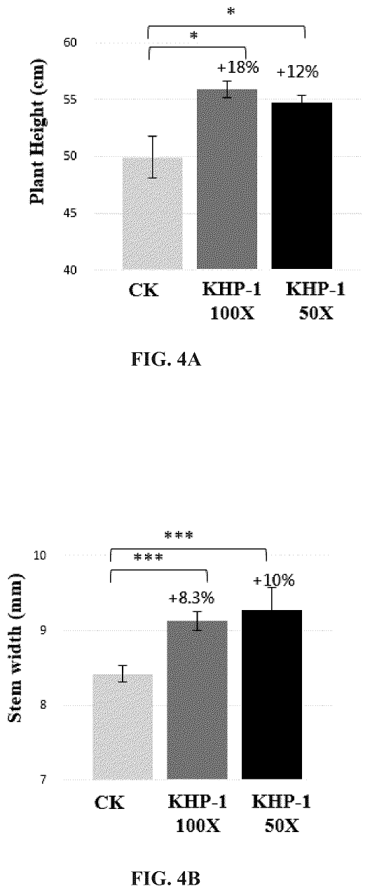

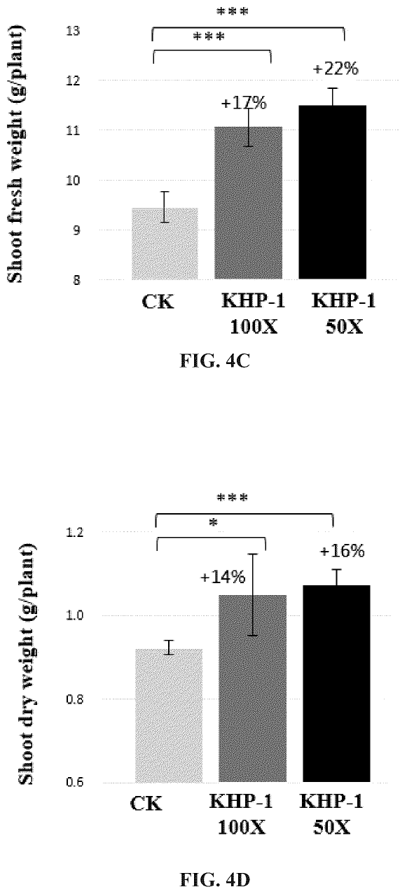

To compare the result on the above-ground stems, the corn seedlings are planted into soil infused with the KHP-1 solutions, both 100× and 50× versions. After 14 days, measurements of the stem's heights, widths, net weights and dry weights are compared and noted in FIGS. 4A, 4B, 4C and 4D respectively.

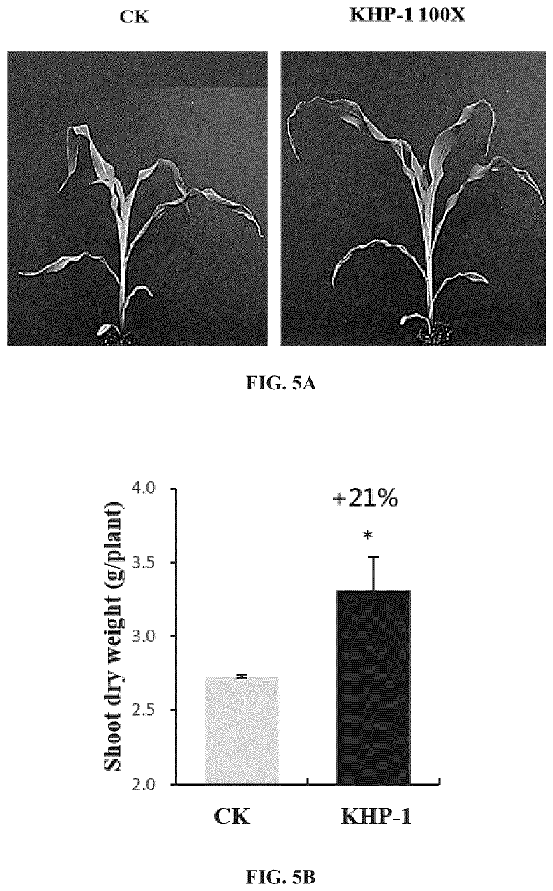

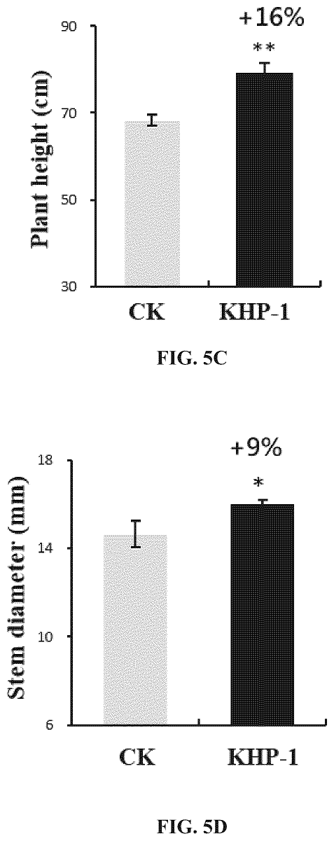

At the young shoot (seedling V3-V4) stage, the KHP-1 100× solution is applied to the corn plants, with the comparisons between the check (CK) group and the KHP-1 group noted in FIGS. 5A, 5B, 5C and 5D, that show, respectively, the differences of growth image, above-ground dry weight, overall height, and stem width.

As can be seen, the KHP-1 group corn plants perform substantially better than that of the check group.

As shown above, different measures taken, at different stages of application confirmed the effectiveness of the KHP-1 solution to promote the growth and development of corn crops.

While the disclosure herein gave limited teachings and embodiment examples, it should be noted that the description and disclosure made herein illustrated the preferred embodiments of the invention and are not meant to limit the scope of the applicant's rights. Variations and alterations may be employed for yet additional embodiments without departing from the scope of the invention herein.

1. A method of using a keratin hydrolysis peptide (KHP) solution to promote the growth and development of corn crops, comprising the steps of:
a. Preparing the KHP solution by mixing 66 kg of feathers whose content is 50% water and 44 kg of water in a sealed container;
b. hydrolyzing the mixture in the container with a temperature and pressure setting of 195° C. and 16 kg/cm² for a duration of 40 minutes;
c. using a mass spectrometer to confirm the combination of peptides in the solution to contain at least 253 peptides as listed in the specification where their molecular masses are between 500 and 4,000 Daltons, and the concentration is in the range of 2.0×10⁵˜4.5×10⁵ ppm;
d. applying the solution to the soil around corn crops at seedling stage.

2. The method of using a keratin hydrolysis peptide (KHP) solution of claim 1 where the solution is sprayed to the corn crops at early growth stage.

3. The method of using a keratin hydrolysis peptide (KHP) solution of claim 1 where the solution is diluted with water by volume at the ratio of 1:50-1000 for applying to the soil or sprayed to the corn crops at the early growth stage.

4. The method of using a keratin hydrolysis peptide (KHP) solution of claim 1 where the solution is diluted with water by volume at the ratio of 1:100-500 for applying to the soil or sprayed to the corn crops at the early growth stage.
